(19) United States
(12) Patent Application Publication
(10) Pub. No.: US 2024/0091514 A1
(43) Pub. Date: Mar. 21, 2024

(54) Transdermal Double-layer Microneedle Based on Hydrogen Producing Probiotics and Its Preparation Method and Application

(71) Applicants: Tangyi Biomedical (Tianjin) Co., Ltd., Tianjin (CN); Tangyi Holding (Shenzhen) Co., Ltd., Shenzhen (CN)

(72) Inventors: Bin ZHENG, Tianjin (CN); Qinglu GUO, Tianjin (CN); Yulin CAO, Beijing (CN); Shixiang CHENG, Tianjin (CN); Zhihui BAI, Beijing (CN)

(73) Assignees: Tangyi Biomedical (Tianjin) Co., Ltd., Tianjin (CN); Tangyi Holding (Shenzhen) Co., Ltd., Shenzhen (CN)

(21) Appl. No.: 17/967,943

(22) Filed: Oct. 18, 2022

(30) Foreign Application Priority Data
Sep. 16, 2022 (CN) 202211134754.7

(51) Int. Cl. A61M 37/00; A61K 9/00; A61K 31/728; A61K 35/741; A61K 38/47; A61K 47/42; A61L 31/04; A61L 31/16

(52) U.S. Cl. CPC A61M 37/0015; A61K 9/0021; A61K 31/728; A61K 35/741; A61K 38/47; A61K 47/42; A61L 31/042; A61L 31/16; C12Y 302/01035; A61K 2035/115; A61M 2037/0023; A61M 2037/0046; A61M 2037/0053; A61M 2037/0061

(57) ABSTRACT

A transdermal double-layer microneedle based on hydrogen producing probiotics and its preparation method and application thereof are provided. The transdermal double-layer microneedle consists of a needle body and a backing layer. The raw material of the needle body includes hyaluronic acid and hyaluronidase. The raw material of the backing layer includes methacrylate anhydride gelatin and silk fibroin protein. The hydrogen producing bacteria are loaded in the microneedles, and the pore size of the matrix material is lower than the diameter of the probiotics, which will not lead to the leakage of bacteria and has high safety. Treatment of inflammatory skin diseases by gas has low side effects. The needle tip is loaded with hyaluronidase to increase the tissue space and improve the effect of gas treatment. It is expected to be used in psoriasis and other inflammatory skin diseases.

### CROSS REFERENCE TO THE RELATED APPLICATIONS

This application is based upon and claims priority to Chinese Patent Application No. 202211134754.7, filed on Sep. 16, 2022, the entire contents of which are incorporated herein by reference.

### TECHNICAL FIELD

The invention belongs to the field of microbial technology, in particular relates to a transdermal double-layer microneedle based on hydrogen producing probiotics and its preparation method and application thereof.

### BACKGROUND

Soluble microneedles are widely used in drug delivery and treatment because of their advantages of easy preparation and painless administration. Insulin can be delivered by microneedle to regulate blood glucose. The use of microneedles to deliver antitumor drugs, accurately locate the position of action, so as to achieve the treatment of tumors; The delivery of vaccines through microneedles can replace the traditional syringe injection to a certain extent and improve the acceptances of patients. The application of endothelial growth factor in wound healing can be realized by microneedle loading. However, the field of microbial microneedle research still needs further study.

The mechanism of inflammation in inflammatory skin diseases is related to oxidative stress. Inflammation can cause aging of epithelial cells and release ROS, and excessive ROS can cause oxidative stress; ROS can promote the synthesis of inflammatory factor TNF-α by upregulating NF-κB signaling pathway. TNF-α can activate the expression of triphosphopyridine nucleotide (NADPH) oxygenase, thereby promoting the synthesis of ROS by NADPH and further aggravating the body's inflammatory response. Hydrogen can regulate the oxidative stress level of cells. By reducing the level of lipid peroxidation products, the activities of Glutathione peroxidase (GSH-Px), catalase (CAT), superoxide dismutase (SOD) and other antioxidant enzymes were increased. Furthermore, apoptosis can be reduced or blocked, thereby blocking the vicious cycle of inflammation and oxidative stress in inflammatory skin diseases.

Traditional inflammatory dermatosis drugs are divided into non-hormonal and hormonal drugs. Non-hormone drugs have defects such as slow onset and relatively poor effect. Although hormone drugs have fast onset, they may still cause local skin atrophy, local infection, or hormone-dependent dermatitis. Microneedles are of interest for their painless delivery, but the small space between the skin tissue limits the diffusion of drug and gas molecules, thus reducing the therapeutic effect. Secondly, the large pore size of the existing microneedle matrix may lead to bacterial leakage and thus cause infection.

### SUMMARY

In view of this, the present invention aims to provide a transdermal double-layer microneedle based on hydrogen producing probiotics and its preparation method and application. The present invention provides a transdermal double-layer microneedle with high safety, low side effects and good therapeutic effect.

In order to achieve the purpose of the invention, the invention provides the following technical scheme:
The invention provides a transdermal double-layer microneedle based on hydrogen producing probiotics, which is composed of a needle body and a backing layer;
The raw material of the needle body comprises hyaluronic acid and hyaluronidase;
The raw material of the backing layer includes methacrylate anhydride gelatin and silk fibroin protein.

Preferably, the hydrogen producing probiotics include Enterobacter aerogens.

Preferably, the aperture of the backing layer is 5-10 μm.

Preferably, the length of the needle body is 200 μm, and the length, width and height of the backing layer are 1 cm, 1 cm and 0.2 cm respectively.

The invention also provides a preparation method of the transdermal double-layer microneedle described in the technical scheme, including the following steps:
1) The mixture was obtained by mixing the 20% hyaluronic acid solution, 30% hyaluronic acid solution and 40% hyaluronic acid solution with the hyaluronidase solution. The mixed liquid is loaded into the needle body part of the PDMS microneedle mold, and the needle body is dried and formed;
2) Gelatin solution was obtained by mixing gelatin with photoinitiator and sonication;
3) The protein solution was obtained by mixing D-sorbitol with silk fibroin solution;
4) The gelatin solution obtained in step 2) was mixed with the protein solution obtained in step 3) and hydrogen producing probiotics. After UV irradiation, the gelatin solution was placed at the back end of the microneedle obtained in Step 1) and dried and formed to obtain the transdermic double-layer microneedle.

Preferably, the volume ratio of 20% hyaluronic acid solution, 30% hyaluronic acid solution and 40% hyaluronic acid solution to hyaluronidase solution in the described step 1) is 1-100:1 respectively;

The enzyme activity of the hyaluronidase solution is 400-10000 U/mg.

Preferably, the photoinitiator of step 2) includes 2-methyl-2-acrylic anhydride;

The photoinitiator is mixed with methacrylate anhydride gelatin in the form of a solution, and the weight volume percentage content of the photoinitiator in the solution is 0.1-10%;

The volume ratio of the methacrylate anhydride gelatin to the solution is 1˜100:1.

Preferably, the mass ratio of D-sorbitol to silk fibroin in step 3 is 1:0.1-10;

The concentration of the silk fibroin solution is 0.01-100 g/mL.

Preferably, the volume ratio of gelatin solution to protein solution in step 4 is 1:1;

The volume ratio of the gelatin solution to the number of hydrogen producing probiotics is 100 μL: 1×10²-1×10¹⁰ CFU.

The invention also provides the application of the transdermal double-layer microneedles described in the technical scheme in the preparation of drugs for the treatment of inflammatory skin diseases.

The invention provides a transdermal double-layer microneedle consisting of a needle body and a backing layer. The matrix material of the backing layer is a mixture of GelMA and silk fibroin. The double-cross-linked matrix has a smaller pore size and supports hydrogen-producing probiotics (Enterobacter aeroglycogens). Limited by the pore size of the substrate material, bacteria produce hydrogen in the microneedles, which act as a gas signaling molecule to regulate inflammation and other functions. The substrate material of needle body was hyaluronic acid and hyaluronidase. After being inserted into the skin, hyaluronidase is dissolved and released into the skin, which enlarges the pore size of the tissue and is more conducive to the diffusion of hydrogen molecules, so as to play a therapeutic role. The preparation is at room temperature, and the hyaluronidase activity is very low, so the enzymatic hydrolysis is only a small part. When applied to the skin, hyaluronidase will be dissolved, leading to the release of hyaluronidase, and the increase of temperature will increase the enzyme activity.

Beneficial effects of the invention are as follows:
(1) The hydrogen producing bacteria are loaded in the microneedles, and the pore size of the matrix material is lower than the diameter of the probiotics, which will not lead to the leakage of bacteria and has high safety;
(2) Treatment of inflammatory skin diseases by gas has low side effects;
(3) The needle tip was loaded with hyaluronidase to increase the tissue space and improve the effect of gas treatment;
(4) It is expected to be used in psoriasis and other inflammatory skin diseases.

### BRIEF DESCRIPTION OF THE DRAWINGS

FIG. 1 shows the gas production of Enterobacter aerogens.

FIG. 2 shows the confocal image of microneedle laser.

### DETAILED DESCRIPTION OF THE EMBODIMENTS

The invention provides a transdermal double-layer microneedle based on hydrogen producing probiotics, which is composed of a needle body and a backing layer. The raw material of the needle body comprises hyaluronic acid and hyaluronidase; The raw material of the backing layer includes methacrylate anhydride gelatin and silk fibroin protein.

In the present invention, the hydrogen producing probiotics preferably include Enterobacter aerogens. In the invention, the loading capacity of the hydrogen producing probiotics of the transdermal double-layer microneedle is preferably 10⁷ CFU.

In the present invention, the aperture of the backing layer is preferably 5-10 μm. In the invention, the length of the needle body is 200 μm, and the length, width and height of the backing layer are 1 cm, 1 cm and 0.2 cm respectively.

In the present invention, the use method of the transdermal double-layer microneedle preferably includes: direct application to the skin.

The invention also provides a preparation method of the transdermal double-layer microneedle described in the technical scheme, including the following steps:
1) The mixture was obtained by mixing the 20% hyaluronic acid solution, 30% hyaluronic acid solution and 40% hyaluronic acid solution with the hyaluronidase solution. The mixed liquid is loaded into the needle body part of the PDMS microneedle mold, and the needle body is dried and formed;
2) Gelatin solution was obtained by mixing gelatin with photoinitiator and sonication;
3) The protein solution was obtained by mixing D-sorbitol with silk fibroin solution;
4) The gelatin solution obtained in step 2) was mixed with the protein solution obtained in step 3) and hydrogen producing probiotics. After UV irradiation, the gelatin solution was placed at the back end of the microneedle obtained in Step 1) and dried and formed to obtain the transdermic double-layer microneedle.

In the invention, the mixture was obtained by mixing the 20% hyaluronic acid solution, 30% hyaluronic acid solution and 40% hyaluronic acid solution with the hyaluronidase solution. The mixed liquid is loaded into the needle body part of the PDMS microneedle mold, and the needle body is dried and formed. The invention preferably loads 1004, of the mixture onto a PDMS microneedle mold.

In the present invention, the volume ratio of 20% hyaluronic acid solution, 30% hyaluronic acid solution and 40% hyaluronic acid solution to hyaluronidase solution is preferably 1-100:1 respectively, more preferably 10:1. The optimal enzyme activity of the hyaluronidase solution is preferably 400-10000 U/mg. In the present invention, the hyaluronidase hydrolyzes hyaluronic acid, which is composed of D-glucuronic acid and N-acetyl-D-glucosamine linked by β-1, 3 glycosidic bonds to form a disaccharide unit. The disaccharide unit is then connected by β-1, 4-glycosidic bond to form polysaccharide, and hyaluronidase acts on β-1, 4-glycosidic bond to hydrolyze to disaccharide. The invention has no special limitation on the drying forming conditions, and the technical personnel in this field can operate according to the conventional operation.

In the invention, gelatin solution was obtained by mixing gelatin with photoinitiator and sonication. In the present invention, the photoinitiator preferably includes 2-methyl-2-acrylic anhydride. In the present invention, the photoinitiator is preferably mixed with methacrylate anhydrated gelatin in solution form, and the weight volume percentage content of the photoinitiator in the solution is preferably 0.1-10%, more preferably 1%. In the present invention, the preferred volume ratio of the methacrylate anhydrated gelatin to the solution is 1-100:1, more preferably 10:1.

In the invention, the protein solution was obtained by mixing D-sorbitol with silk fibroin solution. In the present invention, the mass ratio of D-sorbitol and silk fibroin is preferably 1:0.1-10, and more preferably 1:5. In the invention, the role of D-sorbitol is to reduce the breaking strength of the microneedle, maintain the appearance of the microneedle, not easy to break in the process of penetration, and more easily penetrate the skin. The mechanism is: The sorbitol inserted between the silk fibroin molecular chains plays the role of plasticizer. When the microneedles are subjected to external force, the silk fibroin molecular chains are more likely to slip. In the present invention, the concentration of the silk fibroin solution is preferably 0.01-100 g/mL, more preferably 1 g/mL.

In the invention, the obtained gelatin solution was mixed with the obtained protein solution and hydrogen producing probiotics. After UV irradiation, the gelatin solution was placed on the back end of the microneedle obtained in Step 1), dried and formed to obtain the transdermic double-layer microneedle.

In the present invention, the volume ratio of the gelatin solution to the protein solution is preferably 1:1. In the present invention, the ratio between the volume of the gelatin solution and the number of hydrogen producing probiotics is preferably 100 μL: 1×10²-1×10¹⁰ CFU, more preferably 100 μL: 10⁷ CFU. In the present invention, the optimal conditions for UV irradiation include irradiation for 5 min under a UV lamp of 365 nm. In the present invention, it is preferable to select 100 μL solution after UV irradiation to dry and form the back end of the obtained microne. The present invention has no special limitation on the conditions of drying and forming, and the technical personnel in this field can operate according to the conventional operation. Under UV illumination conditions, the photoinitiator absorbs light energy to produce free radicals, and then makes GelMA intermolecular bond to form solid phase gel. The sorbitol inserted between the silk fibroin protein molecular chains plays the role of plasticizer. When the microneedles are subjected to external force, the slip between the silk fibroin molecular chains is more likely to occur, so that the breaking strength of the microneedles is decreased. It can keep the appearance of microneedles, and it is easier to pierce the skin, and it is not easy to break in the process of piercing.

The invention also provides the application of the transdermal double-layer microneedles described in the technical scheme in the preparation of drugs for the treatment of inflammatory skin diseases.

The technical solutions provided by the present invention are described in detail below in conjunction with embodiments, but they shall not be construed as limiting the scope of protection of the present invention.

### Embodiment 1

Preparation Method of Transdermal Double-Layer Microneedles Based on Hydrogen Producing Probiotics:

(1) Microneedle Preparation—Needle Tip

Prepare hyaluronic acid solutions with different mass percentage (20%, 30%, 40%), add hyaluronidase solution (500 U/mg), and mix well. The volume ratio of 20% hyaluronic acid solution, 30% hyaluronic acid solution and 40% hyaluronic acid solution to hyaluronidase solution is 10:1 respectively. The mixed solution was loaded into the needle body of PDMS microneedle mold by vacuuming, and the mold was dried at room temperature.

(2) Microneedle Preparation—Backing Layer (Length*Width*Height: 1 cm*1 cm*0.2 cm)

200 μL GelMA solution was mixed with 20 μL GelMA solution containing 1% (w/v) 2-methyl-2-acrylic anhydride, and the mixture was homogenized by ultrasound. Then, the prepared solution was transferred to a 4 ml centrifuge tube cap. The D-sorbitol and silk fibroin solution (1 g/mL) were mixed according to the mass ratio of D-sorbitol/silk fibroin=2/10. The above two solutions were mixed in equal volumes, and Enterobacter aerogenic bacteria (obtained by centrifugation of 1*10⁷ cfu/ml bacterial solution) were added, and irradiated for 5 minutes under UV light at 365 nm. 100 μL of the mixed solution was put on PDMS mold in (1), dried and formed, and the bacteria were evenly dispersed in the microneedle backing layer.

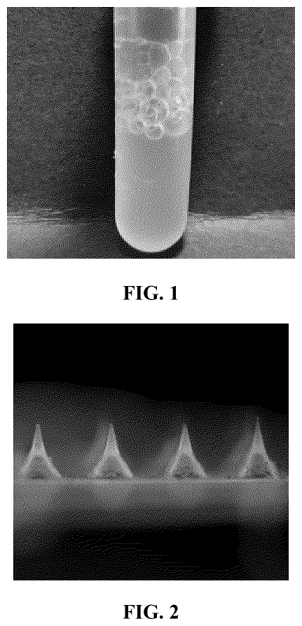

Verification of hydrogen production: After the Enterobacter aerogens were cultured in the medium, the gas production was observed, and the results were shown in FIG. 1.

Microneedle characterization: Nile red fluorescent dye was added in the microneedle preparation process, and then laser confocal photography was performed. FIG. 2 shows the complete shape of the needle.

### Embodiment 2

Preparation Method of Transdermal Double-Layer Microneedles Based on Hydrogen Producing Probiotics:

(1) Microneedle Preparation—Needle Tip

Prepare hyaluronic acid solutions with mass percentage of 20%, add hyaluronidase solution (500 U/mg), and mix well. The volume ratio of 20% hyaluronic acid solution, 30% hyaluronic acid solution and 40% hyaluronic acid solution to hyaluronidase solution is 10:1 respectively. The mixed solution was loaded into the needle body of PDMS microneedle mold by vacuuming, and the mold was dried at room temperature.

(2) Microneedle Preparation—Backing Layer (Length*Width*Height: 1 cm*1 cm*0.2 cm)

200 μL GelMA solution was mixed with 20 μL GelMA solution containing 1% (w/v) 2-methyl-2-acrylic anhydride, and the mixture was homogenized by ultrasound. Then, the prepared solution was transferred to a 4 ml centrifuge tube cap. The D-sorbitol and silk fibroin solution (1 g/mL) were mixed according to the mass ratio of D-sorbitol/silk fibroin=2/10. The above two solutions were mixed in equal volumes, and Enterobacter aerogenic bacteria (obtained by centrifugation of 1*10⁷ cfu/ml bacterial solution) were added, and irradiated for 5 minutes under UV light at 365 nm. 100 μL of the mixed solution was put on PDMS mold in (1), dried and formed, and the bacteria were evenly dispersed in the microneedle backing layer.

### Embodiment 3

Preparation Method of Transdermal Double-Layer Microneedles Based on Hydrogen Producing Probiotics:

(1) Microneedle Preparation—Needle Tip

Prepare hyaluronic acid solutions with mass percentage of 30%, add hyaluronidase solution (500 U/mg), and mix well. The volume ratio of 20% hyaluronic acid solution, 30% hyaluronic acid solution and 40% hyaluronic acid solution to hyaluronidase solution is 10:1 respectively. The mixed solution was loaded into the needle body of PDMS microneedle mold by vacuuming, and the mold was dried at room temperature.

(2) Microneedle Preparation—Backing Layer (Length*Width*Height: 1 cm*1 cm*0.2 cm)

200 μL GelMA solution was mixed with 20 μL GelMA solution containing 1% (w/v) 2-methyl-2-acrylic anhydride, and the mixture was homogenized by ultrasound. Then, the prepared solution was transferred to a 4 ml centrifuge tube cap. The D-sorbitol and silk fibroin solution (1 g/mL) were mixed according to the mass ratio of D-sorbitol/silk fibroin=2/10. The above two solutions were mixed in equal volumes, and Enterobacter aerogenic bacteria (obtained by centrifugation of 1*10⁷ cfu/ml bacterial solution) were added, and irradiated for 5 minutes under UV light at 365 nm. 100 μL of the mixed solution was put on PDMS mold in (1), dried and formed, and the bacteria were evenly dispersed in the microneedle backing layer.

### Embodiment 4

Preparation Method of Transdermal Double-Layer Microneedles Based on Hydrogen Producing Probiotics:

(1) Microneedle Preparation—Needle Tip

Prepare hyaluronic acid solutions with mass percentage of 40%, add hyaluronidase solution (500 U/mg), and mix well. The volume ratio of 20% hyaluronic acid solution, 30% hyaluronic acid solution and 40% hyaluronic acid solution to hyaluronidase solution is 10:1 respectively. The mixed solution was loaded into the needle body of PDMS microneedle mold by vacuuming, and the mold was dried at room temperature.

(2) Microneedle Preparation—Backing Layer (Length*Width*Height: 1 cm*1 cm*0.2 cm)

200 μL GelMA solution was mixed with 20 μL GelMA solution containing 1% (w/v) 2-methyl-2-acrylic anhydride, and the mixture was homogenized by ultrasound. Then, the prepared solution was transferred to a 4 ml centrifuge tube cap. The D-sorbitol and silk fibroin solution (1 g/mL) were mixed according to the mass ratio of D-sorbitol/silk fibroin=2/10. The above two solutions were mixed in equal volumes, and Enterobacter aerogenic bacteria (obtained by centrifugation of 1*10⁷ cfu/ml bacterial solution) were added, and irradiated for 5 minutes under UV light at 365 nm. 100 μL of the mixed solution was put on PDMS mold in (1), dried and formed, and the bacteria were evenly dispersed in the microneedle backing layer.

The foregoing is only a preferred embodiment of the present invention. It should be noted that for ordinary technicians in the technical field, a number of improvements and refinements can be made without deviating from the principles of the invention, and these improvements and refinements shall also be considered as the scope of protection of the invention.

What is claimed is:

1. A transdermal double-layer microneedle based on hydrogen producing probiotics, consisting of a needle body and a backing layer; wherein
a raw material of the needle body comprises a hyaluronic acid and a hyaluronidase;
a raw material of the backing layer comprises a methacrylate anhydride gelatin and a silk fibroin protein.

2. The transdermal double-layer microneedle according to claim 1, wherein the hydrogen producing probiotics comprise Enterobacter aerogens.

3. The transdermal double-layer microneedle according to claim 1, wherein an aperture of the backing layer is 5-10 μm.

4. The transdermal double-layer microneedle according to claim 1, wherein a length of the needle body is 200 μm, and a length, a width and a height of the backing layer are 1 cm, 1 cm and 0.2 cm, respectively.

5. A preparation method of the transdermal double-layer microneedle according to claim 1, comprising the following steps:
1) obtaining a mixture by mixing a 20% hyaluronic acid solution, a 30% hyaluronic acid solution, and a 40% hyaluronic acid solution with a hyaluronidase solution, loading the mixture into a needle body part of a PDMS microneedle mold, and drying and forming to obtain the needle body;
2) obtaining a gelatin solution by mixing the methacrylate anhydride gelatin with a photoinitiator, and performing a sonication;
3) obtaining a protein solution by mixing D-sorbitol with a silk fibroin protein solution;
4) mixing the gelatin solution obtained in step 2) with the protein solution obtained in step 3) and the hydrogen producing probiotics to obtain a resulting mixture, after a UV irradiation, placing the resulting mixture at a back end of the needle body of the transdermal double-layer microneedle obtained in step 1) for drying and forming to obtain the transdermic double-layer microneedle.

6. The preparation method according to claim 5, wherein a volume ratio of the 20% hyaluronic acid solution, the 30% hyaluronic acid solution, and the 40% hyaluronic acid solution to the hyaluronidase solution in step 1) is 1-100:1;
an enzyme activity of the hyaluronidase solution is 400-10000 U/mg.

7. The preparation method according to claim 5, wherein the photoinitiator of step 2) comprises 2-methyl-2-acrylic anhydride;
the photoinitiator is mixed with the methacrylate anhydride gelatin in a form of a solution, and a weight volume percentage content of the photoinitiator in the solution is 0.1-10%;
a volume ratio of the methacrylate anhydride gelatin to the solution is 1-100:1.

8. The preparation method according to claim 5, wherein a mass ratio of the D-sorbitol to the silk fibroin protein solution in step 3 is 1:0.1-10;
a concentration of the silk fibroin protein solution is 0.01-100 g/mL.

9. The preparation method according to claim 5, wherein a volume ratio of the gelatin solution to the protein solution in step 4) is 1:1;
a volume ratio of the gelatin solution to a number of the hydrogen producing probiotics is 100 μL: 1×10²-1×10¹⁰ CFU.

10. A method of an application of the transdermal double-layer microneedle according to claim 1 in a preparation of drugs for the a treatment of inflammatory skin diseases.

11. The preparation method according to claim 5, wherein the hydrogen producing probiotics comprise Enterobacter aerogens.

12. The preparation method according to claim 5, wherein an aperture of the backing layer is 5-10 μm.

13. The preparation method according to claim 5, wherein a length of the needle body is 200 μm, and a length, a width and a height of the backing layer are 1 cm, 1 cm and 0.2 cm, respectively.

14. The method according to claim 10, wherein the hydrogen producing probiotics comprise Enterobacter aerogens.

15. The method according to claim 10, wherein an aperture of the backing layer is 5-10 μm.

16. The method according to claim 10, wherein a length of the needle body is 200 μm, and a length, a width and a height of the backing layer are 1 cm, 1 cm and 0.2 cm, respectively.
